(19) United States
(12) Patent Application Publication
(10) Pub. No.: US 2007/0149509 A1
(43) Pub. Date: Jun. 28, 2007

(54) Use of vitamin e

(76) Inventors: Luca Barella, Basel (CH); Gerald Rimbach, Kiel (DE)

Correspondence Address: NIXON & VANDERHYE, PC, 901 NORTH GLEBE ROAD, 11TH FLOOR, ARLINGTON, VA, 22203, US

(21) Appl. No.: 10/582,906

(22) PCT Filed: Dec. 9, 2004
(86) PCT No.: PCT/EP04/14000

§ 371 Date: Jul. 14, 2006

(30) Foreign Application Priority Data
Dec. 18, 2003 (EP) 03 029 122.3

(51) Int. Cl. A61K 31/5513; A61K 31/4745; A61K 31/405; A61K 31/355; A61K 31/198

(52) U.S. Cl. 514/221; 514/458; 514/419; 514/561; 514/303

(57) ABSTRACT

Vitamin E is useful for facilitating the synchronization of circadian rhythm in humans, especially for treatment or prevention of jet lag.

The present invention relates to a novel use of vitamin E. More particularly, the present invention relates to a method of facilitating the synchronization of circadian rhythm in humans, especially to a method of treating or preventing jet lag, which method comprises administering to a person in need of such treatment or prevention an effective amount of vitamin E, optionally in combination with an agent known to synchronize the circadian rhythm. In a further aspect the present invention relates to the use of vitamin E, optionally in combination with an agent known to synchronize the circadian rhythm, in the manufacture of a composition for facilitating the synchronization of circadian rhythm in humans, especially treatment or prevention of jet lag. In still another aspect, the present invention relates to novel-compositions comprising vitamin E and an agent known to synchronize the circadian rhythm.

The term “vitamin E” as used herein refers to all tocopherols, i.e. α-, β- and γ-tocopherol in all steric forms, as well as to physiologically acceptable esters thereof such as the acetates, although racemic α-tocopherol and natural vitamin E and their acetates are preferred for the purpose of the present invention.

The mammalian circadian clock in the brain conveys 24-hr rhythmicity to sleep-wake cycles, temperature, locomoter activity and virtually all other behavioral and physiological processes. In order for these cycles to be adaptive, they must be synchronized, or entrained, to the 24-hr light/dark cycle produced by the rotation of the Earth. Air travelers who cross several time zones are commonly affected by jet-lag symptoms which include impaired sleep, mood and cognitive performance which result from the body's internal rhythms being out of step with the day-night cycle at the destination. Circadian rhythm sleep disorders are a group of pathologies characterized by an internal de-synchronization between a person's biological clock and their environmental 24-hr schedule. Winter depression and delayed sleep phase syndrome (DSPS) belong to this class of disorders. In the context of this invention, the phrase “facilitating the synchronization of circadian rhythms” also comprises a method of assisting shift workers to adjust to rotating work schedule, and a method of treating sufferers of delayed spleen phase syndrome (DSPS) and winter depression.

Critically timed exposure to bright light and melatonin administration can help to reduce symptoms. Bright light is one of the powerful synchronizers of human rhythms and melatonin serves as a “dark pulse” helping to induce nighttime behaviors. Melatonin is a pineal hormone and its potential clinical implication in treatment of pathological and/or induced circadian disorders is well recognized. Melatonin acts via three melatonin receptors, MT1-MT3. It has been shown that overexpression of MT1 do significantly increase the biological effect of melatonin.

It has unexpectedly been found that the melatonin receptor type 1 gene (MT1) is strongly and consistently up-regulated in the brains of vitamin E supplemented animals.

Two groups of male rats were randomly assigned to either a vitamin E deficient diet or to a diet containing vitamin E for 290 days. High-density oligonucleotide microarrays comprising over 7,000 genes were used to assess the transcriptional response of the brain. Differential gene expression was monitored over a period of 9 months, at 4 different time-points, and rats were individually profiled (FIG. 1).

Accordingly, administration of vitamin E, optionally in combination with an agent known to synchronize the circadian rhythm, facilitates the synchronization of circadian rhythms in humans and, thus, is useful in the treatment and prevention of jet lag symptoms. Optionally, an agent known to synchronize the circadian rhythm may be co-administered in a dosage known to be effective for said agent. Examples of agents known to synchronize the circadian rhythm are melatonin, coffein, benzodiazepines, e.g., Temazepan or Triazolam, glycine, cycloprones or imidazopyridines, e.g., Zolpidem. Of particular interest for the purposes of the present invention is the combination of vitamin E with melatonin.

An appropriate dosage regimen for treating and/or preventing jet lag symptoms in accordance with the presence invention is the administration of about 10 International Units (IU) to about 1000 IU to an adult person per day, suitably one week before and 4 days after day-night chance occurrence. If agents known to synchronize the circadian rhythm are co-administered such agents are administered in dosages known to be effective for that purpose. Thus, melatonin may be co-administered with vitamin E in a dosage of from about 5 mg per day according to the above regimen.

Vitamin E and, optionally, further agents known to synchronize the circadian rhythm may be administered as a pharmaceutical formulation, e.g., as a drageé or capsule, or in a food or beverage. Agents for co-administration with vitamin E in accordance with the present invention can be administered in separate formulations or together with vitamin E in a single formulation.

The impact of vitamin E on melatonin receptor expression in rat hippocampus was determined as follows:

Two groups of male rats were randomly assigned to either a VE sufficient diet or to a diet deficient in VE for 270 days. High-density oligonucleotide microarrays comprising over 7,000 genes were used to assess the transcriptional response of the brain. Differential gene expression was monitored over a period of 9 months, at 4 different time-points. The results are shown in FIG. 1below.

[image]

The impact of vitamin E and placebo on jet lag symptoms in humans may be determined as follows: 200 volunteers who will have flights over 6 to 8 time zones are involved in a double-blind, randomized, placebo-controlled study. The volunteers receive either vitamin E 500 UI or placebo. The study substance is taken once daily at bedtime during 1 week before and 4 days after an eastward flight. The volunteers are to complete the Profile of Mood States (POMS), sleep log and symptoms questionnaires once daily and the Karolinskia Sleepiness Scale (KSS) three time daily prior to departure and during the 4 days of vitamin E supplementation postflight [Suhner, A, et al. Chronobiol Int (1998) November; 15(2):655-666.]. The self-rated sleep quality, shortened sleep latency, fatigue and daytime sleepiness is then compared between the vitamin E and the placebo groups.

The following Example illustrates the invention further.

### EXAMPLE

A soft gelatin capsule may be prepared comprising the following ingredients:

| Per capsule | mg |
|---|---|
| Vitamin E ((all-rac)-α-tocopherol) | 200 |
| Melatonin | 5 |

1. The use of vitamin E in the manufacture of a composition for facilitating the synchronization of circadian rhythm in humans, especially for treatment or prevention of jet lag.

2. The use as in claim 1 of vitamin E in combination with an agent known to synchronize the circadian rhythm.

3. The use as in claim 2 wherein said agent is selected from melatonin, a benzodiazepine, glycine, a cycloprone, or an imidazopyridine.

4. The use as in claim 1 wherein the composition is a medicament.

5. The use as in claim 1 which is a food or beverage.

6. A composition comprising vitamin E and an agent known to synchronize the circadian rhythm.

7. A composition comprising vitamin E and melatonin.

8. A composition as in claim 6 which is a pharmaceutical composition.

9. A composition as in claim 8 wherein said pharmaceutical composition comprises about 10 IU to about 1000 IU of vitamin E and about 2 mg to about 5 mg of melatonin per dosage unit.

10. A method of facilitating the synchronization of circadian rhythm in humans, especially of treating or preventing jet lag which comprises administering to an adult person in need of such treatment or prevention from about 10 IU to about 1000 IU of Vitamin E per day, optionally in combination with an agent known to synchronize the circadian rhythm.

11. A method as in claim 10 wherein vitamin E is administered 1 week before and 4 days after day-night change occurrence.

12. A method as in claim 10 wherein melatonin is administered simultaneously.

13. A method as in claim 12 wherein 5 mg of melatonin are administered per day.
